(19) United States
(12) Patent Application Publication
(10) Pub. No.: US 2020/0113502 A1
(43) Pub. Date: Apr. 16, 2020

(54) APPARATUS AND METHOD FOR EVALUATING OTOLITH DYSFUNCTION

(71) Applicant: Rajneesh BHANDARI, Jaipur (IN)

(72) Inventors: Rajneesh BHANDARI, Jaipur (IN); Anita BHANDARI, Jaipur (IN)

(21) Appl. No.: 15/575,388

(22) PCT Filed: Sep. 4, 2015
(86) PCT No.: PCT/IB2015/056758

§ 371 Date: Nov. 20, 2017

(30) Foreign Application Priority Data
May 26, 2015 (IN) 1501/DEL/2015

(51) Int. Cl. A61B 5/00

(52) U.S. Cl. CPC A61B 5/4023; A61B 5/6803

(57) ABSTRACT

The present invention provides an apparatus for evaluating otolith dysfunction through the subjective horizontal and vertical testing (SHVT) of a patient. The present apparatus includes a projector adapted to project a luminous line on a test wall of a test room at an initial predetermined position, a controller operated by a patient that adjusts the position of luminous line w.r.t. patient's input. The said input is analyzed by a computing system included in present apparatus which computes subjective visual parameters of patient based on the said input. The subjective visual parameters are calculated by distinguishing deviation of the luminous line (patient's input) from the predetermined reference position. This reference position is installed in the computing device and not visible to the patient. Further, the projector is adapted to add a static, dynamic or real life background.

### FIELD OF THE DISCLOSURE

The present invention relates to the field of evaluating and diagnosing otolith dysfunction of a person. More particularly, the present invention provides an apparatus and method for determining subjective visual vertical (SVV) and subjective visual horizontal (SVH) values of a person.

### BACKGROUND OF THE DISCLOSURE

The Otolith organ of the vestibular system is located in the structure of the inner ear, and is responsible for the horizontal and vertical orientations by sensing gravity and linear accelerations to give humans a sense of balance. Vertigo is a symptom arrived from pathologies in vestibular system.

One known method of conducting SHVT test for the diagnosis of otolith dysfunction discloses a “Light bar method”. In the Light bar method, the patient is seated upright with the head restrained or unrestrained 1.5 meters in front of a wall of the test room. The test room in this case is a dark test room. Then the patient is instructed to gaze at a dim light bar (30×1 cm) which is mounted on the wall of the test room. Prior to testing, the examiner positions the bar at an oblique angle. During testing, the light bar is rotated by the examiner about its center axis according to the patient's verbal instruction until the bar reaches the patient's perception of verticality (“as it would coincide with a plumb line”).

The above light bar method has many drawbacks as the light bar casts a shadow on the wall of the test room. Moreover, the light and the background may give a visual clue to the patient about the orientation of the light bar and thus defeats the purpose of the test. Secondly, the angle has to be calculated manually which is prone to error. Further, the examiner moves the light bar as per the directions of the patient which means the examiner has to stand near the light bar. Hence, the examiner's vertical position gives a clue to the patient and defeats the purpose of the test. If the patient moves the light bar, then he gets a clue due to gravity.

Another known method of conducting SHVT test for otolith testing is the “hemispheric dome” method. In “hemispheric dome” method, the patient is allowed to sit with his chin resting on a fixed pad and looking into a hemispheric dome. The dome is 60 cm in diameter and completely fills one's visual field. The dome is characteristically covered with a random pattern of colored dots, providing no cues to gravitational orientation. There is a linear target placed inside the dome at least thirty centimeters apart in front of the patient. The centre of such linear target is fixed on the shaft of a computer-controlled servomotor. During the test, such linear target is rotated in the patient's frontal plane according to examiner specifications. Following random rotation of such linear target from vertical, the patient is instructed to align the linear target with his or her perceived vertical using a joystick device. Differences between the patient's adjusted orientation and true spatial vertical are calculated by the examiner using the system computer taking the average of 10 readjustments. Accordingly, subjective visual vertical (SVV) of the patient is determined.

Other known test for evaluating otolith dysfunction discloses a “Bucket” test. In this method, the patient is allowed to sit upright and look into a translucent plastic bucket, wherein, the visual field of the patient is covered completely by the rim of the bucket. There is a dark, straight, diametric line placed on the bottom inside of the bucket. On the bottom outside of the bucket, there is an arrow connected by a shaft with the line placed on the bottom inside of the bucket. The said arrow originates from center point of the bottom of the bucket and said arrow points towards degree angles as marked on the bottom rim of the bucket. Accordingly, the angle of deviation between the patient's perceptual position and the actual position is measured.

While conducting the bucket test, the said bucket is randomly rotated clockwise or counterclockwise by the examiner to various end positions and then slowly rotated back to the zero degree position. This rotation of the bucket by the examiner excludes the haptic clues. When the patient estimate that the inside bottom line of the bucket is in vertical position, then the patient raises a signal to stop the movement of the bucket. Then the examiner measures the degree of angle difference between the inside bottom line and the outside bottom line. This test can be performed by asking the patient to keep both eyes open (binocular) and with one eye covered (monocular left/right).

The above provided tests for evaluating otolith dysfunction have various drawbacks such as vertical and horizontal clues to the patient, involvement of many manually operated hardware devices, manual measuring the angle difference between the perceptual vertical/horizontal position and actual vertical/horizontal position, and overall cost of the method. Moreover, the “hemisphere” and “bucket” testing method has their own problems such as they very are cumbersome tests with a hemisphere and a bucket respectively thrust in the face of the patient. Furthermore there are a lot of moving parts which make them prone to breakdown. Moreover, the system is rather costly and the whole test procedure is time consuming. Also, none of the apparatus used to carry the tests as described above are able to provide real life backgrounds.

Hence, there is a need to develop a simple, efficient and cost effective apparatus which can efficiently evaluate otolith dysfunction or vestibular disorders through the known in the art subjective horizontal and vertical testing (SHVT) of the patient.

### SUMMARY

The present invention provides an apparatus and method adapted to evaluate otolith dysfunction quickly, accurately and cost effectively through known in the art testing the subjective visual horizontal testing and subjective visual parameters of the patient.

The invention includes a projector, a controller and a computing system. The projector and the controller are operationally connected to the computing device. The projector projects a luminous line on a test wall of a test room at an inclined predetermined position and the patient is asked to align the said inclined luminous line in different positions such as vertical position and horizontal position. To align the said luminous line, the patient uses the controller as provided to him.

The said controller is adapted to change the position of the luminous line on the test wall of the test room. Further, the computing system as provided herein is adapted to measure and analyze a plurality of patient subjective visual parameters. These patient subjective visual parameters includes subjective visual vertical and subjective visual horizontal. The said plurality of patient subjective visual parameters is measured by determining the angle of deviation between the patient's perceptual position of the luminous line and the actual reference position of the luminous line. The said actual reference position of the luminous line is preloaded in the computer system and not visible to the patient.

In an embodiment, the present invention includes an eye wear unit to be worn by the patient while conducting the present otolith dysfunction test. The said eye wear unit includes tubular shaped projections emerging out of the glasses, wherein the tubular shaped projections are adapted to create a tubular vision to the patient. The tubular vision to the patient is very helpful for cutting down the external clues with regard to the actual vertical and actual horizontal positions of the test environment.

In a preferred embodiment, the present invention provides an apparatus and method for evaluating the subjective visual vertical of the patient. The said subjective visual vertical is evaluated by measuring the deviation angle of the luminous line from the actual vertical reference position to the perceptual vertical position.

In a preferred embodiment, the present invention provides an apparatus and method for evaluating the subjective horizontal vertical of the patient. The said subjective visual horizontal is evaluated by measuring the deviation angle of the luminous line from the actual horizontal reference position to the perceptual horizontal position.

It is an objective of the present invention to build very simple and user friendly diagnostic apparatus and technique to be used by individuals of any age group and individual having different body structure such as height, size of the head, and weight.

Another object of the present invention is to employ a technique involving minimum physical activity as possible. Accordingly, the apparatus and the method of the present invention is very helpful for evaluating the otolith dysfunction of the peoples who cannot perform intensive physical activity as required in prior arts.

Another object of the present invention is to save the test time and produce the reports of the subjective horizontal and vertical test (SHVT) results as quickly as possible.

Yet another object of the present invention is to provide a simple vestibular disorder test procedure which is inexpensive.

These and other objects and advantages of the invention will be clear from the ensuing description.

### DESCRIPTION OF THE DRAWINGS

The advantages and features of the present invention will become better understood with reference to the following detailed description taken in conjunction with the accompanying drawings, in which:

FIG. 1 illustrates a traditional method for evaluating otolith dysfunction using hemispheric dome method;

FIG. 2 illustrates another traditional method for evaluating otolith dysfunction using bucket method;

FIG. 3 illustrates a block diagram showing the present innovative apparatus and method to evaluate otolith dysfunction, according to various embodiments of the present invention;

FIG. 4 illustrates a test room 200 including the present innovative apparatus and method to detect otolith dysfunction of a patient 202 through SHV and SVV testing, according to various embodiments of the present invention;

FIG. 5a illustrates a front view of an eye wear unit comprising a pair of tubular shaped glasses, in accordance with an embodiment of the invention;

FIG. 5b illustrates a side view of an eye wear unit comprising a pair of tubular shaped glasses, in accordance with an embodiment of the invention;

FIG. 6a illustrates a test illustration of the projection of the luminous line at an inclined position, according to an embodiment of the present invention;

FIG. 6b illustrates a test illustration of the projection of the luminous line at a predetermined vertical/horizontal reference position for calculating angle of deviation, according to an embodiment of the present invention;

FIG. 7a illustrates a test illustration of the angle of deviation between a perceptual horizontal position and an actual horizontal reference position, according to an embodiment of the present invention;

FIG. 7b illustrates a test illustration of the angle of deviation between a perceptual vertical position and an actual vertical reference position, according to an embodiment of the present invention;

FIGS. 8 and 9 illustrate a test illustration of the projection of the luminous line in a dynamic background and a real life background, according to an embodiment of the present invention; and

FIG. 10 illustrates a head mounted projection and display system for projecting the luminous line and the background, according to another embodiment of the present invention.

Like numerals depict like elements throughout the description.

### DESCRIPTION OF THE INVENTION

The exemplary embodiments described herein detail for illustrative purposes are subjected to many variations. It should be emphasized, however, that the present invention is not limited to device and method for evaluating otolith dysfunction. It is understood that various omissions and substitutions of equivalents are contemplated as circumstances may suggest or render expedient, but these are intended to cover the application or implementation without departing from the spirit or scope of the present invention.

The terms “a” and “an” herein do not denote a limitation of quantity, but rather denote the presence of at least one of the referenced item.

The terms “having”, “comprising”, “including”, and variations thereof signify the presence of a component.

The term “known arts”, “prior arts” or “earlier arts” used herein should not be construed as “prior arts”, rather understood as terms used for illustrative purposes.

The term “patient” denotes any subject person or human being undergoing the diagnosis.

As used herein, the term “SVV” stands for subjective visual vertical, “SVH” stands for subjective visual horizontal, “SVHT” stands for subjective horizontal and vertical testing.

The diagnosis of the otolith dysfunction is quite costly and lacks efficiency. As disclosed in earlier arts, like in the light bar method where SVV is tested that assess the perception of the gravitational vertical by visualizing an illuminated rod or bar which can rotate in its midpoint along the horizontal axis by the examiner according to the patient's verbal instructions. The test is done in a completely dark room, so the subject has no external visual cues besides the rod, which is initially set in a random position (random tilt angle). The objective is to align the bar or rod to the gravitational vertical by rotating it. No feedback other than the visual feedback should be present.

The problem with this method is that, the light bar casts a shadow in the background which may give a hint regarding the orientation and defeats the purpose of the test. Further, the examiner moves the bar as per instructions of the patient which amounts to certain human error on the examiner's end, also he would have to stand close to the light bar resulting in a chance where the patient gets an idea of examiner's vertical position which can be used to orient the bar, hence, defeating the purpose of the test.

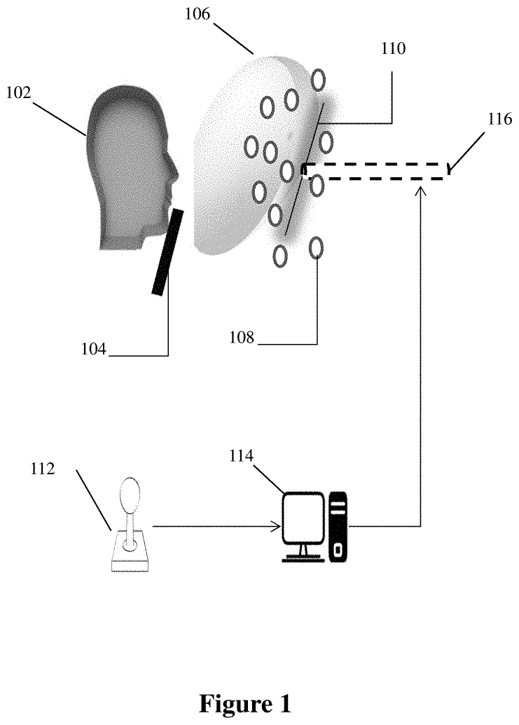

A prior hemispheric dome method as illustrated in FIG. 1 depicts a patient 102 whose chin rests on a fixed pad 104 and patient 102 faces the hemispheric dome 106 of 60 cm diameter completely filling the patient's visual field. The dome 106 is characteristically covered with a random pattern of colored dots 108, providing no cues to gravitational orientation. Thirty centimeters in front of the patient 102 is a linear target 110 whose center is fixed on the shaft of a servomotor 116 controlled by a computer 114. During the test, the target 110 is rotated in the patient's frontal plane according to examiner specifications with the help of a joystick 112. Differences between the patient's adjusted orientation and true spatial vertical are calculated by the examiner using the system computer taking the average of 10 readjustments. SVV is determined binocularly. The dome 106 being thrust in patient's face and presence of additional components for the diagnosis make it complex and as the dome method consists of various moving parts it is prone to breakdown.

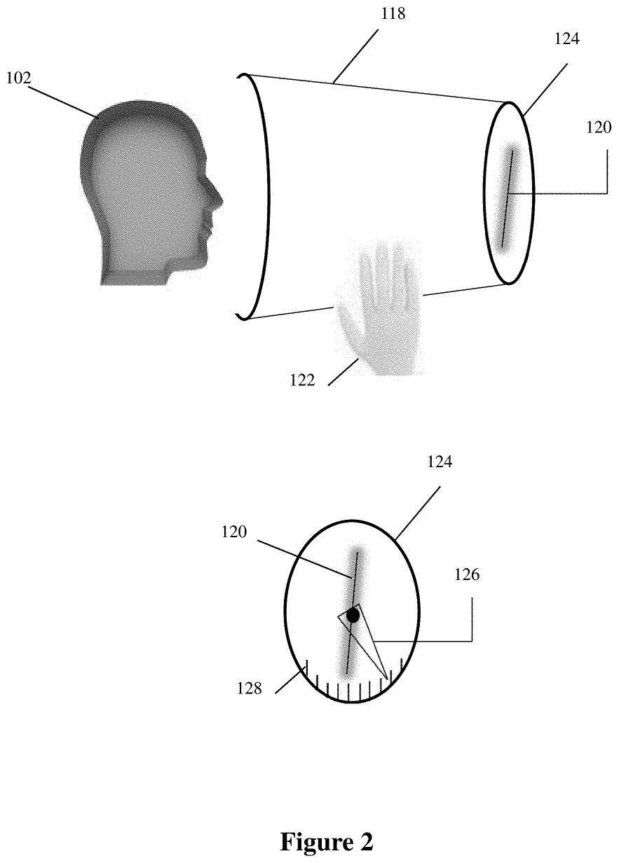

Similarly, in bucket method as illustrated in FIG. 2, the patient 102 sits upright and looks into a translucent plastic bucket 118 while the examiner holds the bucket through his hand 122; the visual field is covered completely by the rim of the bucket. On the bottom 124, inside the bucket, there is a dark, straight, diametric line 120. On the bottom 124, outside there is an arrow 126 connected with the line 120 by a shaft. The bottom 124 is divided into degrees 128 from outside. Patients signal when they estimate the inside bottom line to be truly vertical by saying “stop.” Degrees are read off on the outside scale by the examiner. Measurements can be made with both eyes open (binocular) and with one eye covered (monocular left/right). Like the hemisphere dome method, this method too has a structure i.e. bucket 118 thrust in patient's face, also the examiner needs to hold the bucket through his hand 122, rotate the line 120 according to patient's input and stop when the patient feels that the line has been aligned, whereby making the system prone to human error.

Therefore, a device and a method is needed which could calculate the otolith dysfunction while removing the drawbacks such as lack of efficiency, complexity and cost effectiveness. The innovative apparatus described herein utilizes a projector, controller and a computing system, whereby making the method of evaluating otolith dysfunction cost affordable and efficient. The device has no moving parts, it is not based on verbal communication with patient which may be prone to errors, and should be inexpensive. Therefore, the present invention provides an apparatus which allows performing of the conventional SHVT tests.

The present innovative apparatus and method involves a projector to project a luminous line on a wall of a test room. The orientation of the line is controlled by the patient through a remote controller. The said controller is connected to the projector which in turn is connected to a computing system. The computing system is preloaded with actual horizontal and vertical parameters and compares these preloaded parameters with patient's perceptual vertical or horizontal alignment of the line. Accordingly, the computing system measures the angle of deviation and evaluates the otolith dysfunction. As the invention utilizes the patient's input and projects it directly without any human intermediate, hence the room for human error is reduced to zero. Also, the present invention uses projector, remote controller and computer as the only hardware devices which are easily available and can be easily coupled with each other. Accordingly, the present invention reduces the cost and time of diagnosis of otolith dysfunction.

The claims and various aspects of the invention will be apparent after the following description of figures.

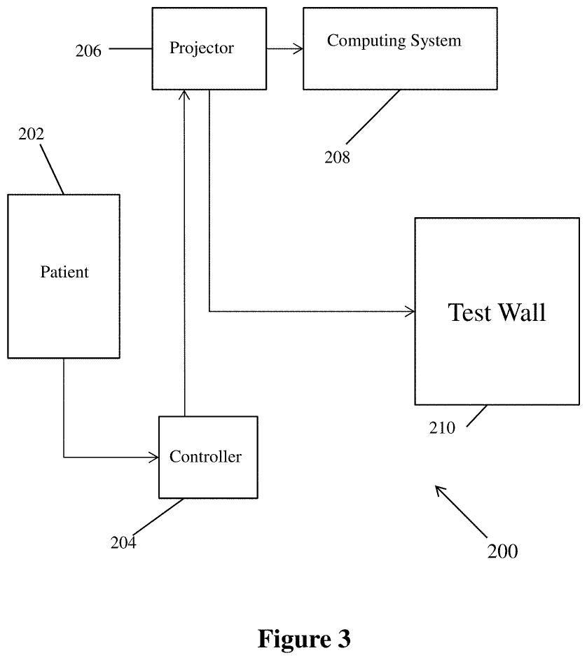

A patient 202 is allowed in a test room 200 as illustrated in the block diagram shown in FIG. 3. The projector 206 projects a luminous line on the test wall 210 of the test room 200. The orientation of the luminous line can be controlled by a controller 204 given to patient. The projector 206 is operationally coupled with a computing system 208 for the evaluation of otolith dysfunction.

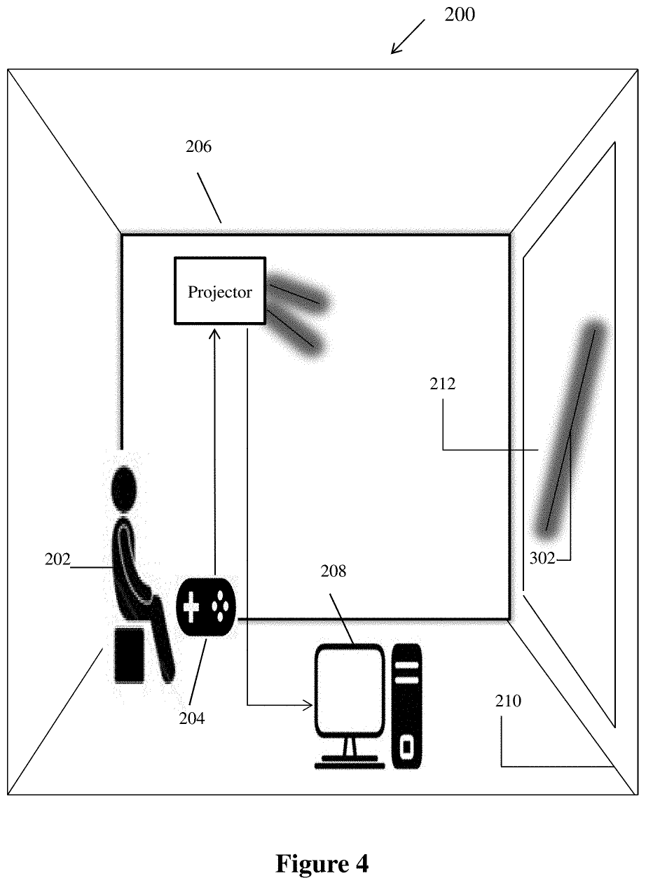

Now referring to FIG. 4, there is illustrated the test scenario for the diagnosis of otolith dysfunction using the present inventive apparatus and method. The patient 202 in a test room 200 faces a test wall 210. The projector 206 projects a luminous line 302 onto the test wall 210. The orientation of projection is initially predetermined by the computing system 208 or by the examiner (not shown). In another implementation, the test wall 210 includes a test screen 212, which occupies all or some portion of the wall 210 where the line 302 is projected. The projection of the projector 206 is adjusted by the controller 204 handed over to the patient 202. In an implementation, the patient 202 could be seated or standing during the diagnosis.

The controller 204 is a controlling device such as a remote, joystick etc. which alters the orientation of the line 302. The control 204 is connected to the projector 206 by wire or wirelessly. In an implementation, the controller 204 has a joystick which is rotated to adjust line orientation. In another implementation, the controller 204 has buttons for adjusting the line orientation. In yet another implementation, the controller 204 has option of a rotating wheel to adjust the orientation of line 302. The controller 204 can be a combination of any or all implementations mentioned above.

When the diagnosis begins, the luminous line 302 is projected at a specified angle or initial predetermined position at the test wall 210. The examiner then asks the patient 202 to align the line 302 either vertically or horizontally to calculate the SVV or the SVH parameters using the controller 204. The SHVT is performed on the basis of the alignment of the line 302 using the angle of deviation between the line 302 with respect to actual horizontal or vertical lines which are preloaded in the computing system 208 and not visible to the patient. After the patient is done adjusting the line 302, the computer calculates the angle of deviation and generates a report based on the calculations of patient subjective visual parameters. In another implementation, the subjective visual parameters are utilized by the examiner to generate diagnostic report.

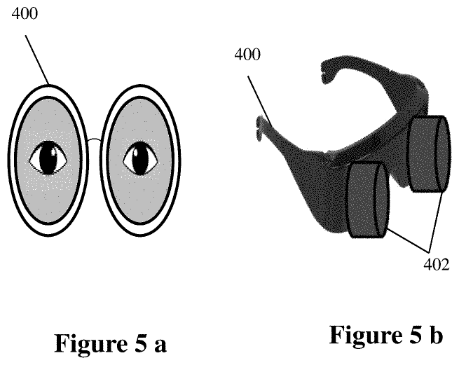

FIGS. 5a and 5b demonstrate, in accordance with an embodiment of invention, an eye wear unit 400, a device adapted to be worn, strapped or placed around the eyes of the patient 202 during the diagnosis. The eye wear unit 400 has a tubular vision which narrows down the visual field of the patient 202 to eliminate any visual clues from the edge of the walls. The eye wear unit 400 as shown in FIG. 5a illustrates a front view and FIG. 5b illustrates a side view. In an implementation, the eye wear unit 400 consists of cylindrical structures 402 protruding from either viewing apertures. The structures 402 not necessarily being cylindrical in geometry could be in any shape that blocks the side view for the patient 202 by converging the his/her visual field to the projection on the test wall 210.

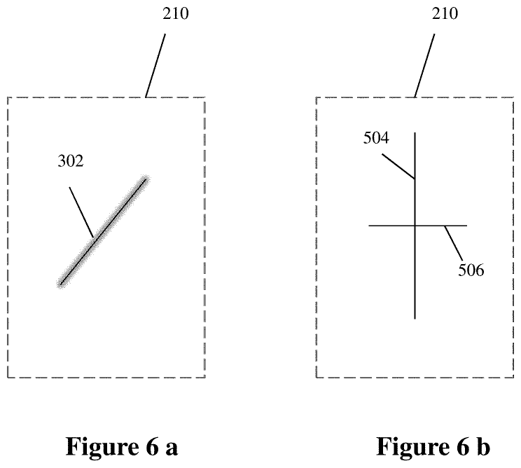

Originally in the diagnosis the luminous line 302 is set on the test wall 210 in a predefined position or angle determined by the examiner or randomly positioned by the computing system 208 as depicted in FIG. 6a. An actual reference position as depicted in FIG. 6b consisting actual horizontal 506 and actual vertical 504 references are preinstalled in the computing system 208 which are used to determine the angle of deviation or subjective visual parameters. The co-ordinates of the actual reference position are fixed or aligned with the center of the luminous line 302 and are adapted as per the line 302 is moved in the x and/or y (horizontal and/or vertical) plane. The actual reference position is not known or invisible to the patient 202.

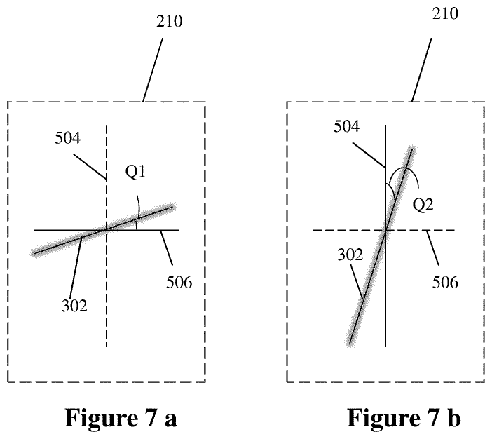

When the patient 202 is asked to align the line 302 along the horizontal (x) axis, the patient 202 does so with the help of controller 204 and aligns the line 302 as per his perception of horizontal axis. This is depicted in FIG. 7a where the orientation of line 302 is carried out by the patient according to what he/she perceives as horizontal axis. The difference between the perceived horizontal 302 and actual horizontal 506 is represented by Q1. The angle of deviation Q1 is subjective horizontal visual parameter or SVH. The actual vertical 504 is dashed just to highlight its void during evaluation of SVH in the test scenario.

When the patient 202 aligns the line 302 vertically as depicted in FIG. 7b, the angle between his/her perceived vertical 302 and actual vertical 504 represented by Q2 is the subjective vertical visual parameter or SVV. The actual horizontal 506 is dashed to represent its void during the evaluation of SVV in the test scenario. The SHVT is a combination of the above mentioned SHV and SVV which is used by innovative apparatus and method to determine otolith dysfunction.

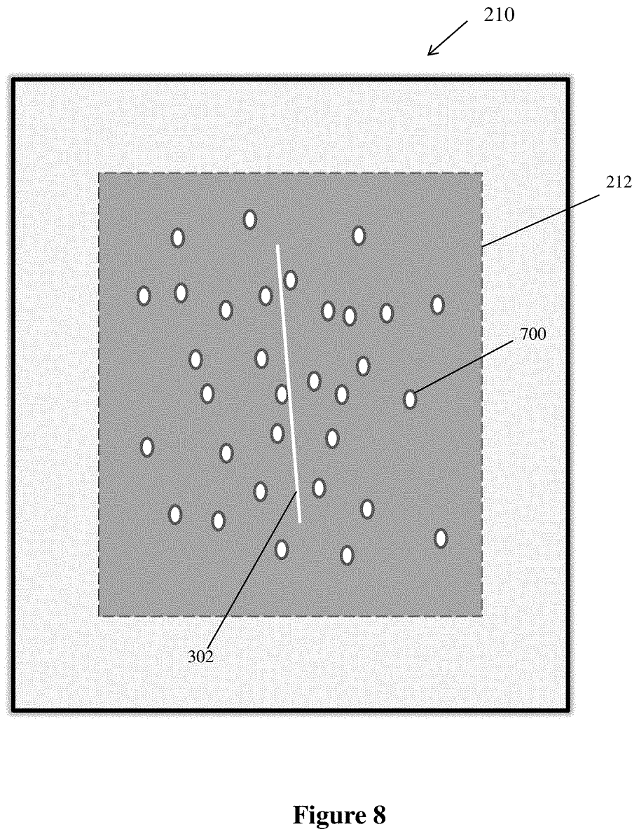

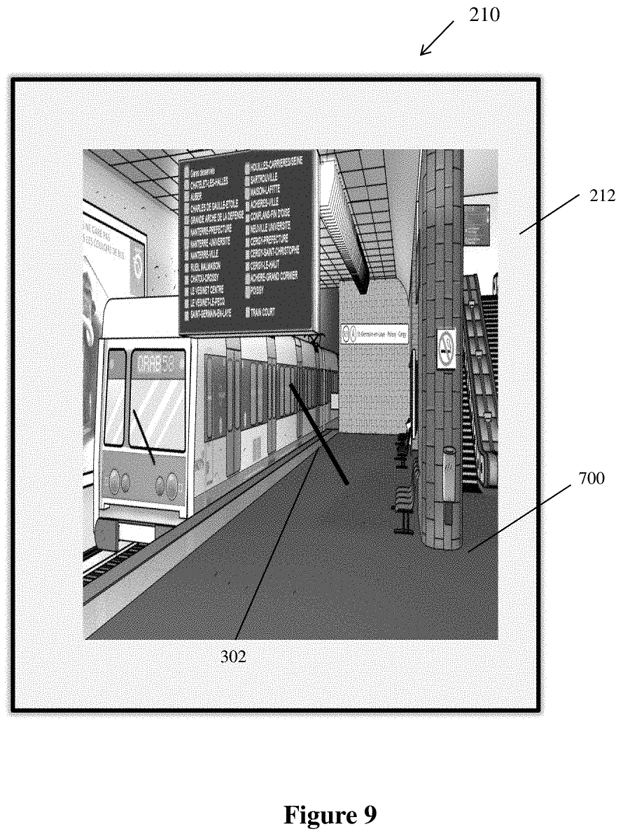

FIG. 8 depicts, in an embodiment of the invention, SHVT in dynamic background and real life background. The dynamic background testing is another way for diagnosis of otolith dysfunction which has a background, here test wall 210, lit with colored static or moving structures 700, as shown in FIGS. 8 and 9. The real life background allows an examiner to introduce real life scenes and situations in the testing procedure by introducing real life images (static or moving) of the real life scenes and situations. The luminous line 302 is projected and same procedure is followed as mentioned above to evaluate subjective visual parameters. In an implementation the dynamic testing is performed by placing a test screen 212 on whole or a portion of test wall 210.

The computing system 208 can be a normal computer or special dedicated computer using software for evaluating subject visual parameters. The software calculates the angle of deviation between the subject perceptual horizontal/vertical and actual horizontal/vertical parameters. The test wall 210 or background for the invention is dark or colored based on static or dynamic testing.

The patient 202 is seated on a chair in a dark room 200 2.5 m from the test wall 210. An LCD projector 206 is used to project a luminous line 302 on the test wall 210. This line 302 can be rotated with a remote clicker 204 which is given to the patient 202. The patient 202 is made to wear specially designed goggles 400 ensuring tubular vision and cut off of peripheral cues. The position of the line 302 is inclined at the beginning of the test (FIG. 6a) and then the patient 202 is asked to make it vertical using the remote clicker 204. The software then calculates the angle of the line 302 to actual vertical (FIG. 7b). This test is repeated starting with the line inclined to left and then right with a static background (test wall 210). It is then done with a dynamically moving background (FIG. 8)—first clockwise, followed by counterclockwise movement. The speed of rotation of the background (test wall 210) is 40 degree/sec. This test is repeated in the same way with the patient 202 being given instructions to make the line horizontal. At the end of each test, angulation of the line is measured. A deviation up to 2.5 degree is considered within normal range. Deviation beyond 2.5 degrees is an indicator of otolith dysfunction.

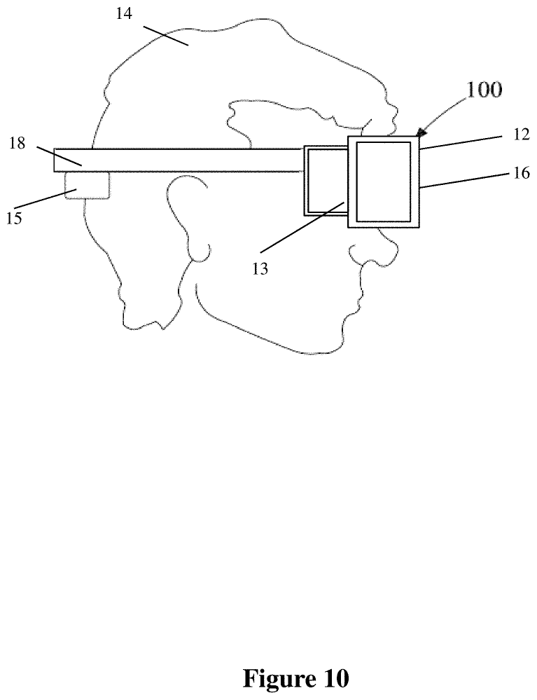

FIG. 10 depicts yet another embodiment of the present invention. More specifically, FIG. 10 illustrates a head mounted projection and display apparatus 100. The apparatus 100 includes a frame 12 adapted to be worn by a patient 14. More specifically, the apparatus 100 includes a band 18 extending from the frame 12. The patient 14 can wrap around the frame 12 over his/her head using the said band 18 and secure the frame 12 thereon using securing element 15, like Velcro, and the like.

The frame 12 further includes a projector 13 embodied in the frame 12. The projector 13 is adapted to project the luminous line. The apparatus 100 may include required microelectronics for ensuring the projection of the luminous line in this manner

Further, the frame 12 includes a screen 16 built integral to the frame 12 and embodied therein. The screen 16 is used to have the projection of the luminous line thereon. The screen 16 may be made of suitable materials and may include necessary microelectronics to enable projection of the luminous line thereon. The apparatus 100 may further include a controller (not shown) operable by the patient 14 for changing position of the luminous line on the screen 16, and a computing system adapted for measuring and analyzing at least a plurality of patient subjective visual parameters, wherein the plurality of patient subjective visual parameters are based on deviation of position of the luminous line from a predetermined reference position when operated by the patient. The functioning of the controller and the computing system is similar to the explanation of controller and the computing system as mentioned above.

Therefore, as per this embodiment, the luminous line is projected on the screen 16 right in front of the patient 14. This precludes the need of having a test wall, such as test wall 210. Accordingly, the apparatus 100 is designed to be compact, handy and portable. Further, the head mounted projection and display apparatus 100 has the advantage of not requiring a dark room.

The present innovative apparatus and method has no moving parts such as servomotor in hemispheric dome method (FIG. 1) and involves no human error as in light bar method or bucket testing method (FIG. 2). The invention is most cost effective requiring no hardware except computer and projector. The invention is repeatable, reliable and easy for the doctor and patient to perform and the report is automatically generated. Moreover, various dynamic backgrounds and real life images which simulate the conditions faced by the person in daily life can be used in this test. For example, multiple dots moving towards the person simulate numerous people coming out of a crowded station as perceived by the person standing outside the station.

Further, the present invention should not be construed to be limited to the configuration of the method and system as described herein only. Various configurations of the system are possible which shall also lie within the scope of the present invention.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The embodiments were chosen and described in order to best explain the principles of the present invention and its practical application, and to thereby enable others skilled in the art to best utilize the present invention and various embodiments with various modifications as are suited to the particular use contemplated. It is understood that various omissions and substitutions of equivalents are contemplated as circumstances may suggest or render expedient, but such omissions and substitutions are intended to cover the application or implementation without departing from the spirit or scope of the present invention.

The computing system 208 executes software or a set of instructions that are stored in one or more storage elements in order to process input data. The storage elements may also hold data or other information as desired. The storage element may be in the form of an information source or a physical memory element present in the processing machine.

The set of instructions may include various commands that instruct the processing machine to perform specific tasks such as the steps that constitute the method of the disclosed teachings. The set of instructions may be in the form of a software program. The software may be in various forms such as system software or application software. Further, the software might be in the form of a collection of separate programs, a program module with a larger program or a portion of a program module. The software might also include modular programming in the form of object-oriented programming. The software program or programs may be provided as a computer program product, such as in the form of a computer readable medium with the program or programs including the set of instructions embodied therein. The processing of input data by the processing machine may be in response to user commands or in response to the results of previous processing or in response to a request made by another processing machine.

What is claimed is:

1. An apparatus for evaluating otolith dysfunction through the subjective horizontal and vertical testing (SHVT) of a patient, the apparatus comprising:
at least one projector adapted to project a luminous line on a test wall of a test room at an initial predetermined position;
a controller operable by the patient for changing position of the luminous line on the test wall; and
a computing system adapted for measuring and analyzing at least a plurality of patient subjective visual parameters,
wherein the plurality of patient subjective visual parameters are based on deviation of position of the luminous line from a predetermined reference position when operated by the patient.

2. The apparatus as claimed in claim 1, wherein the projector is operationally connected to the computing system, and the controller is operationally connected to the projector.

3. The apparatus as claimed in claim 1 further comprising an eye wear unit adapted to be worn by the patient, wherein the eye wear unit comprises a pair of tubular shaped frame.

4. The apparatus as claimed in claim 3, wherein the pair of tubular shaped fame of the said eye wear unit is adapted to create a tubular vision to the patient.

5. The apparatus as claimed in claim 1, wherein an operator sets the luminous line at an inclined position with reference to the test wall at the beginning of the SHVT test.

6. The apparatus as claimed in claim 5, wherein the patient remotely operates the projector via the controller to move the luminous line from an inclined position to a perceptual vertical position.

7. The apparatus as claimed in claim 6, wherein the computing system is adapted to measure the deviation angle of the luminous line from the actual vertical reference position to the perceptual vertical position.

8. The apparatus as claimed in claim 7, wherein the patient remotely operates the projector via the controller to move the luminous line from an inclined position to a perceptual horizontal position.

9. The apparatus as claimed in claim 8, wherein the computing system is adapted to measure the deviation angle of the luminous line from the actual horizontal reference position to the perceptual horizontal position.

10. The apparatus as claimed in claim 5, wherein the luminous line is inclined to a left side, or to a right side over the test wall.

11. The apparatus as claimed in claim 5, wherein the luminous line is inclined with a static background or with a dynamically moving background or a real life background over the test wall.

12. The apparatus as claimed in claim 11, wherein the dynamically moving background comprises a background having a plurality of small light dots, wherein the plurality of small light dots are moving in a clockwise direction or in an anticlockwise direction.

13. The apparatus as claimed in claim 12, wherein the plurality of small light dots is moving at a speed of 20-50 degree per second.

14. The apparatus as claimed in claim 12, wherein the real life background comprises a background having images of real life scenes and situations.

15. A method for evaluating otolith dysfunction through the subjective horizontal and vertical testing (SHVT) of a patient, the method comprising:
projecting a luminous line on a test wall of a test room through at least one projector;
allowing the patient to change position of the luminous line on the wall via a controller; and
computing at least a plurality of patient subjective visual parameters through a computing system,
wherein the plurality of patient subjective visual parameters are based on deviation of position of the luminous line from an actual reference position when operated by the patient.

16. The method as claimed in claim 15 comprises allowing the patient to wear an eye wear unit having a pair of tubular shaped frame, wherein the pair of tubular shaped frame of the said eye wear unit is adapted to create a tubular vision to the patient.

17. The method as claimed in claim 15 comprising allowing the patient to sit on a test chair positioned towards the test wall of the test room, wherein the test chair is positioned 2-10 meter away from the test wall.

18. The method as claimed in claim 16 comprising setting the luminous line at an inclined position with reference to the test wall at the beginning of the SHVT test.

19. The method as claimed in claim 18, wherein the patient remotely operates the projector via the controller to move the luminous line from the inclined position to a perceptual vertical position during the test.

20. The method as claimed in claim 19, wherein the computing system computes the deviation angle of the luminous line from the actual vertical reference position to the perceptual vertical position.

21. The method as claimed in claim 18, wherein the patient remotely operates the projector via the controller to move the luminous line from the inclined position to a perceptual horizontal position during the test.

22. The method as claimed in claim 19, wherein the computing system computes the deviation angle of the luminous line from the actual horizontal reference position to the perceptual horizontal position.

23. The method as claimed in claim 20, wherein the luminous line is inclined to a left side, or to a right side over the test wall.

24. The method as claimed in claim 20, wherein the luminous line is inclined with a static background, or with a dynamically moving background or with a real life background over the test wall.

25. The method as claimed in claim 24, wherein the dynamically moving background comprises a background having a plurality of small light dots, wherein the plurality of small light dots are moving in a clockwise direction or in an anticlockwise direction.

26. The method as claimed in claim 24, wherein the real life background comprises a background having images of real life scenes and situations.

27. A head mountable apparatus for evaluating otolith dysfunction through the subjective horizontal and vertical testing (SHVT) of a patient, the apparatus comprising:
a frame having,
a projector configured in the frame adapted to project a luminous line at an initial predetermined position,
a screen integral to the frame, the screen adapted to receive projection made by the projector;
a controller operable by the patient for changing position of the luminous line on the test wall; and
a computing system adapted for measuring and analyzing at least a plurality of patient subjective visual parameters,
wherein the plurality of patient subjective visual parameters are based on deviation of position of the luminous line from a predetermined reference position when operated by the patient.

28. The apparatus as claimed in claim 27, wherein an operator sets the luminous line at an inclined position with reference to the glass wall at the beginning of the SHVT test.

29. The apparatus as claimed in claim 28, wherein the patient remotely operates the projector via the controller to move the luminous line from an inclined position to a perceptual vertical position.

30. The apparatus as claimed in claim 29, wherein the computing system is adapted to measure the deviation angle of the luminous line from the actual vertical reference position to the perceptual vertical position.

31. The apparatus as claimed in claim 27, wherein the luminous line is inclined with a static background or with a dynamically moving background or a real life background over the screen.

32. The apparatus as claimed in claim 31, wherein the dynamically moving background comprises a background having a plurality of small light dots, wherein the plurality of small light dots are moving in a clockwise direction or in an anticlockwise direction.

33. The apparatus as claimed in claim 32, wherein the real life background comprises a background having images of real life scenes and situations.
